(19) United States
(12) Patent Application Publication
(10) Pub. No.: US 2021/0331107 A1
(43) Pub. Date: Oct. 28, 2021

(54) FILTRATION ARTICLE FOR PERSONAL PROTECTIVE EQUIPMENT

(71) Applicant: Ion Defense Technologies, LLC, Hamilton, MT (US)

(72) Inventors: Mark Holler, Hamilton, MT (US); David Morrow, Waco, TX (US)

(21) Appl. No.: 17/239,344

(22) Filed: Apr. 23, 2021

Related U.S. Application Data
(60) Provisional application No. 63/015,893, filed on Apr. 27, 2020.
(60) Provisional application No. 63/048,559, filed on Jul. 6, 2020.

(51) Int. Cl. B01D 39/08; B01D 39/16; B32B 5/02; B32B 5/26; B32B 5/24; A41D 13/11; A62B 23/02

(52) U.S. Cl. CPC B01D 39/086; B32B 2571/00; B01D 39/1623; B32B 5/022; B32B 5/024; B32B 5/026; B32B 5/26; B32B 5/245; A41D 13/1138; A62B 23/025; B01D 2239/0442; B01D 2239/0609; B01D 2239/0613; B01D 2239/0435; B01D 2239/0428; B01D 2239/0407; B01D 2239/065; B01D 2239/0258; B01D 2239/0618; B32B 2262/106; B32B 2262/02; B32B 2266/0264; B32B 2264/1051; B32B 2264/1055; B32B 2307/7145; B32B 2307/724; B32B 2307/73; B01D 39/1676

(57) ABSTRACT

A filtration article for using in personal protective equipment (“PPE”) that includes one or more layers. The article has an external side for facing side the environment and an internal side for facing the body of a user. The article is air permeable but has a porosity sufficient to entrap and thereby filter selected infectious agents (IAs). The one or more layers comprise structures providing two or more of the following functional features selected from the group of: (1) a structure with oligodynamic materials for inactivating the selected IAs; (2) a structure of carbon fibers or particles for moisture management and/or conductive cooling; (3) a knit or woven structure having yarns in a denier gradient that causes wicking of water from the internal side of the mask toward the external side; (4) an energized or energizable structure that subjects entrapped IAs to current, charged particles, electrostatic discharge, resistive heating, IR heating and/or disruptive electromagnetic energy; and (5) a hydrophobic structure forming an external surface of the external side for repelling droplets and other moisture from absorbing into the surface.

### BACKGROUND

The inventive subject invention relates generally to a filtration article, which is useful in personal protective equipment (“PPE”) that serves to filter air from an exterior side of the article to a body facing side of the article. For example, the inventive subject matter may be used as a protective face mask or respirator, a buff, a bandage or wound covering, and similar uses. More particularly, the filtration article incorporates oligodynamic materials. Basically, oligodynamic metals that destroy or operationally disrupt infectious, pathogenic biological agents like bacteria, fungi, spores, and viruses, disabling their infectious nature (hereinafter, any such agent may be referred to as an “IA”. The inventive subject matter is especially suitable for use as a face mask or respirators, which will be used hereinafter as a representative article of personal protective equipment.

Facial masks have been used for hundreds of years to protect medical workers and people in close contact with others when there is danger of infection.

Masks are crucially needed in hospitals, residential care facilities, workplaces, sporting events, concerts, large shopping centers, airplanes and public transportation. They are needed by people who are healthy to diminish potential infection from breathing the same air or receiving spray (i.e. sneeze). They also protect those around a person who is infected but asymptomatic.

In 2020, Hong Kong, a city of 7.5 million people, instituted mandatory wearing of masks and social distancing very early in the COVID-19 pandemic. The city infection rate was very low compared to places that did not take early precautions. There is a world-wide need for masks for the general public.

A traditional mask (e.g. 3M Particulate Respirator 8210) seals effectively around the nose and mouth and is comfortable enough to wear for extended periods. A traditional mask works by filtering particles and droplets. For example, a N95 mask filters 95% of fine particles. Traditional masks are made from cotton and are considered one-use. Traditional masks do not kill or neutralize IAs, nor do they wick moisture and heat away from the user. Unfortunately, they are the most commonly available. Worldwide there is a limited production capacity for one-use traditional masks. This invention described here is a product that can be washed and reused many times and can be mass produced using existing technology for rapid supply expansion. Testing for such masks is described by ASTM F2100-11 and is incorporated herein by reference.

Various designs and configurations for face masks have been previously proposed. One class of masks uses a filter network to trap the pathogens. These face masks include the surgical type masks commonly worn in hospitals. One example is described in U.S. Pat. No. 7,044,993 to Bolduc entitled “Microbicidal air filter.” Bolduc discloses a system that employs an immobilization network of fibers having antimicrobial agents incorporated and molecularly bonded into its structure. Another class of masks include those that employ filter canisters to trap the pathogens. One example is described in U.S. Pat. No. 6,681,765 to Wen entitled “Antiviral and antibacterial respirator mask.” Wen discloses a system that employs a filtration apparatus containing both an active stage and passive stage filter in the mask.

Many metals are known to have oligodynamic action. For instance, silver has been a known antibiotic agent for at least 6,000 years. It was used to store food and prevent spoiling in ancient Babylon, as evidenced by archaeological finds. Silver has been used in wound dressings for many decades and was particularly important before the discovery of penicillin. Silver fabrics are used by the military to prevent fungus and bacterial infection on the battlefield in such products as bandages, socks and underwear. Copper is a well-known oligodynamic element, either in raw form or combined with other metals (e.g., brass). Research has shown that copper or brass elements (e.g., bed frames) in hospital environments substantially reduce transmission of IAs. The mechanism for this reduction occurs when bacteria and viruses are destroyed or inactivated by encountering copper or copper compounds.

In recent time, textiles impregnated or treated with very fine silver bits or other oligodynamic particles been developed. In some cases, the particles are on the nanoscale.

Numerous metals and metallic compounds may emit ions that disrupt bacteria via three pathways: 1. Respiration, 2. Replication, and 3. Cell wall synthesis. Likewise, metals or metallic compounds may disrupt viruses by disassociating the fatty membrane surrounding the RNA. The metallic ions also disrupt the proteins that may surround a virus which allow it to attach to and penetrate a living cell. Metals such as copper or silver are much less likely to promote the development of resistant IA than traditional antibiotics that typically target only one of these pathways. Antimicrobial silver has been used extensively in hospitals for decades with no clinically relevant cases of antibiotic resistance.

The H1N1 virus is thought to have caused the 1918 influenza pandemic and swine flu outbreak in 2009. Silver nanoparticles have been shown to inhibit viruses such as H1N1, as cited here: “Our data suggest that silver nanoparticles exert anti-HIV activity at an early stage of viral replication, most likely as a viricidal agent or as an inhibitor of viral entry. Silver nanoparticles bind to gp120 in a manner that prevents CD4-dependent virion binding, fusion, and infectivity, acting as an effective viricidal agent against cell-free virus (laboratory strains, clinical isolates, T and M tropic strains, and resistant strains) and cell-associated virus. Besides, silver nanoparticles inhibit post-entry stages of the HIV-1 life cycle.” Mode of antiviral action of silver nanoparticles against HIV-1 in the Journal of Nanobiotechnology, 2010 Jan. 20. Humberto H Lara, Nilda V Ayala-Nuñez, Liliana Ixtepan-Turrent, and Cristina Rodriguez-Padilla.

Unfortunately, existing PPE filtration articles have various deficiencies that need to be overcome. While some may be effective at entrapping IAs, the IAs may remain in the filter in a dangerous active state. This causes risk to the PPE user handling and using the article. Typically, therefore, the article needs to be discarded after a single or limited use. During use of the articles, particularly masks and respirators, moisture and heat can build-up on the body-facing side. This causes users discomfort. Further, it may cause the articles to degrade faster, which also limits their use to single or limited use. For these and other reasons, there is a substantial need for improved PPE articles.

### SUMMARY

The inventive subject matter overcomes the deficiencies in the prior art by providing a PPE article that not only traps IAs in a filter medium but also provides modes for inactivating the entrapped IAs so that the masks can be more safely used and handled. By reducing the risk from entrapped IAs, the article may be used longer before it needs to be discarded. The article may be constructed from durable materials so that it may be washed or laundered for repeated use. The article may also include functional features for moisture and thermal management on the internal side of the article. The article may include a first line of defense in the nature of a barrier layer on the outer surface of the article to help prevent IAs from entering the article.

The inventive subject matter advantageously combines certain features that provide a multimodal system for preventing IAs from passing through the filtration article, helping to disrupt the IAs, and providing comfort to the PPE user by moisture management and thermal regulation. The filtration article also may be reused. And it can be cost effectively produced.

The inventive subject invention relates generally to a filtration article, which is useful in PPE. It serves to filter air from an exterior or environmental side of the article to a body facing side of the article. For example, the inventive subject matter may be used as a protective face mask or respirator, a buff, a bandage or wound covering, and similar uses.

In one aspect, the inventive subject matter incorporates oligodynamic materials into a layer of material that is included in a PPE article. In another aspect, the inventive subject matter includes an exterior surface that repels environmental droplets and moisture ladened with IAs. In a further aspect, the PPE article may include a moisture management feature. In another aspect, the PPE article may include a thermal regulation feature for conductively dissipating heat generated by the user or otherwise present on the user side of the article. In another aspect, the inventive subject matter may include a feature that kills or disrupts IAs via an energizable layer or layers. Layers may be energized via direct electrical current, electrostatic charge, charged particles, resistive heating, or IR heating.

In some possible embodiments, the invention relates to a protective face mask that cost-effectively offers broad spectrum antimicrobial protection using fabrics treated with one or more oligodynamic metals and/or salt of an oligodynamic metal. The article also incorporates thermal management and water repellency via textile selection and/or incorporation of carbon fibers into the textile. The article may be constructed of common or available materials so that it can be reused 25 times or more.

In one possible embodiment, the inventive subject matter is directed to a filtration article for using in personal protective equipment (“PPE”) that includes one or more layers. The article has an external side for facing side the environment and an internal side for facing the body of a user. The article is air permeable but has a porosity sufficient to entrap and thereby filter selected IAs. The one or more layers comprise structures providing two or more of the following functional features selected from the group of: (1) a structure with oligodynamic materials for inactivating the selected IAs; (2) a structure of carbon fibers or particle for moisture management and/or conductive cooling; (3) a knit or woven structure having yarns in a denier gradient that causes wicking of water from the internal side of the mask toward the external side; (4) an energized or energizable structure that subjects entrapped IAs to current, charged particles, electrostatic discharge, resistive heating and/or disruptive electromagnetic energy;

and (5) a hydrophobic structure forming an external surface of the external side for repelling droplets and other moisture from absorbing into the surface.

The foregoing and other embodiments are described in more detail in the following detailed descriptions and the figures.

The following is a description of various inventive lines under the inventive subject matter. The appended claims, as originally filed in this document, or as subsequently amended, are hereby incorporated into this Summary section as if written directly in.

The foregoing is not intended to be an exhaustive list of embodiments and features of the inventive subject matter. Persons skilled in the art can appreciate other embodiments and features from the following detailed description in conjunction with the drawings.

### BRIEF DESCRIPTION OF THE DRAWINGS

The appended figures show embodiments according to the inventive subject matter, unless noted as showing prior art.

FIGS. 1A-1B schematically show a 2-layer filtration article with a first layer having an internal side against a user's skin and an adjacent outer layer having an external side to open air.

FIGS. 2A-2B schematically show a filtration article implemented as a face mask or respirator, with FIG. 2A showing the article in place on a user's face, the view including an oval cutaway to illustrate the three layers, and with FIG. 2B showing a cross section of the three layers in more detail.

### DETAILED DESCRIPTION

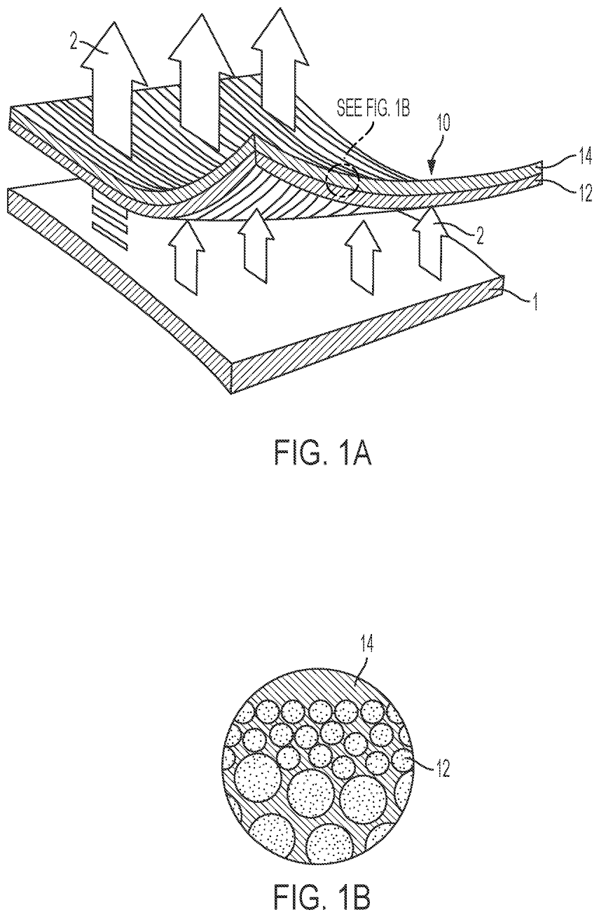

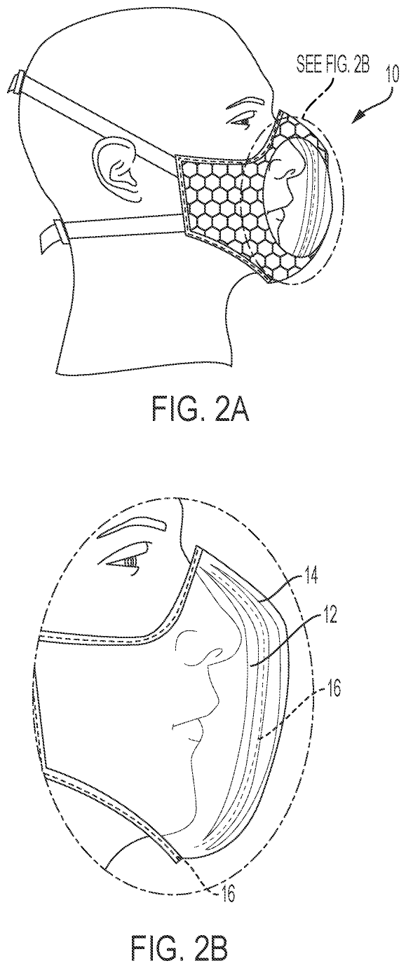

Representative embodiments according to the inventive subject matter are shown in FIGS. 1A-2B, wherein the same or generally similar features share common reference numerals.

In general, the articles of PPE contemplated herein include a first, bodyside or inner layer 12 that is oriented against a user's skin. A second layer 14 is adjacent the first layer or spaced apart from it by one or intermediate layers 16 and is oriented towards the external environment. One or both of the first and second layers generally would have a filtration function for capturing external particles in a porous network. The porosity can be defined to physically exclude particles, including IAs, from passing through to the surface of the body side layer towards the user's skin or inhalation passages, or other body parts needing protection. Filtration media include woven, knit, and non-woven textiles. They may also include non-textiles like polymer foams. The filtration media may be any combination of the foregoing constructs. Filter media is well known in the art and commercially available. For masks and respirators, the porosity and density of the filtration media are controlled to allow for sufficient airflow for respiration while still entrapping particles. Masks and other filtration articles may include mechanical one-way valves to help vent gases 2 and vapor 2 from the bodyside of the article while allowing environmental air to enter.

The inventive subject matter advantageously combines certain functional features that provide a multimodal system for preventing IAs from passing through the filtration article, helping to inactivate the IAs, and providing comfort to the user by moisture management and thermal regulation. The articles are formed from an innovative selection and arrangement of structures that synergistically operate to more effectively protect users and provide the users comfort. Advantageously, a single structure may provide multiple functions. Or, by a scheme of selectively arranging layers, different advantageous effects can be achieved. Certain of the following structures may be selected to achieve desired effects and objectives:
(1) a structure with oligodynamic materials for inactivating the selected IAs;
(2) a structure (e.g., a woven, knit, non-woven textile structure) that includes carbon fibers or particles (which may be referred to as a “carbonized structure”) for moisture management and/or conductive cooling;
(3) a knit or woven structure having yarns in a denier gradient that causes wicking of water from the internal side of the mask toward the external side;
(4) a carbonized structure that is an energized, or energizable, that subjects entrapped IAs to current, charged particles, electrostatic discharge, resistive heating and/or disruptive electromagnetic energy (this structure may be the same as or different from the carbonized structure for moisture/thermal management); and
(5) a hydrophobic structure forming an external surface of the external side for repelling droplets and other moisture from absorbing into the surface.

The filtration articles according to the inventive subject matter may include some or all the foregoing five primary functional features, in any permutations.

In many applications, the structures are woven, knit, or non-woven textile structures. However, other non-textiles may also serve as structures. For example, polymer foams or sheet materials may support functional features contemplated herein. For example, a porous polymer sheet could provide air permeability and have oligodynamic materials associated with it. An open cell foam could similarly serve as a substrate for functional features. By controlling cell size, the foam could also serve as a filtration medium. Polymers and foams can also provide a structural backbone for shaping an article of PPE to a better conform to a body part. Or they can serve as bonding layers interconnecting other layers.

The filtration article also may be constructed so that it may be reused and washed or laundered. And it can be cost effectively produce.

In one possible embodiment, the inventive subject matter is directed to a protective face mask that cost-effectively offers a broad spectrum of antimicrobial protection using fabrics treated with one or more oligodynamic metals and/or salt of an oligodynamic metal. The oligodynamic material may be in the form of a thread, a small particle, an impregnation of fabric threads, or a film.

A first functional feature serves to kill, destroy, disrupt or otherwise make the IA inactive so it cannot harm an intended user. As one example of forming an oligodynamic structure in the article, metallic elements are impregnated into, coated on, or otherwise associated with a fabric so that they can inactivate viruses and bacteria. Numerous tests show a neutralizing capacity of over 99% for 600 bacteria and virus species using certain oligodynamic materials. Articles according to the inventive subject matter may use commercially available fabrics that are treated with a nanoparticle metal solution or incorporate nanoparticles of metal directly into the yarn or fibers where it is woven or knit at a fabric mill.

The nanoparticles can be incorporated into any structure of the article so that a single structure has multiple functional features. For example, a mill could weave or knit the fabric containing both the nano-metal and carbon fiber for the carbonized structure. In either case there will be millions of metallic oligodynamic nanoparticles incorporated into each device.

A second functional feature helps provide comfort to the user and/or helps prevent degradation of the article by managing moisture and heat that may build up on the internal side of the article. For example, in a mask, heat and moisture build up on the internal side as the user exhales. To deal with this, the article may incorporate carbon fiber in yarns that are woven or knit into a fabric used as a structure in the article. Carbon fiber incorporated into a worn garment provides excellent heat and moisture management and has been used in sports clothing for this reason. The user of the article benefits from a more comfortable product and thus would be more likely to wear a mask that incorporates carbon fiber yarns or strands into the fabric.

A third functional feature is another moisture management tool that operates by wicking moisture from the internal side of the article. The article may be constructed with a wicking system. For example, a denier gradient fabric wicks moisture away from the wearer, enhancing user comfort and article durability. Denier gradient fabrics comprise multiple fabric layers having different deniers that scale in one direction. The denier gradient causes moisture to travel by capillary action from the larger denier fabric side to a smaller denier fabric side. U.S. Pat. No. 4,733,546, issued Mar. 29, 1988, to Toda, titled “KNITTED FABRIC FOR CLOTHING,” incorporated herein by reference, describes one such variable denier gradient fabric (“Toda”). In particular, Toda describes a fabric having a surface layer yarn of a certain denier, such as, for example, 1.0 denier to 2.5 denier. The back layer of the fabric would be preferably 50% or more larger than the surface layer denier. The voids between the larger denier fibers of the back layer would be larger than the voids between the smaller denier fibers in the surface layer. Thus, capillary action would cause moisture to move from the back layer towards the surface layer. This action has been found useful in designing moisture management fabrics.

This action is also described in U.S. Pat. No. 6,381,994B1, filed Jun. 28, 2001 by Young-Kyu Lee titled “Method for making fabric with excellent water transition ability”. Refer to FIG. 1 for a drawing of this fabric technology. Patent Number: EP0766520 to Laycock and Walker (expired) describes a technique to create a denier gradient fabric, “A multilayer breathable cloth of a clothing garment, said cloth comprising at least two separate layers interlocked, the layers having differing deniers so as to provide a denier gradient through the thickness of the fabric, at least one of the layers being of a woven structure, wherein the finer denier layer is located at the outside of the garment.” A denier gradient is illustrated in FIGS. 1A-1B, which show layer 12 subdivided into a first layer of a larger gradient and second layer of a smaller gradient. FIGS. 1A-1B also show the movement of water 2 or moisture 2 away from a skin adjacent 1 the first layer 12 to the exterior surface of the second layer 14.

A fourth functional feature provides an energized or energizable structure that helps to inactivate IAs. A carbonized structure can provide a substrate for such function. Carbon fiber is generally anisotropic and conducts electricity in a linear fashion along the length of the carbon fiber as opposed to transversely across the width of the fiber. However, carbon fiber has a potential for a small amount of transverse electrical current flow. Treating fabric (which contains carbon fiber strands) with a solution containing metal nanoparticles will bring the two dissimilar materials into contact. Depending on the anodic index of the metal used there will certainly be some current or ion movement, essentially creating a low-power battery or electrostatic effects. We believe that this electrical potential may substantially enhance the oligodynamic effectiveness of the mask because ion exchange is known to disrupt bacteria and viruses. Other possibilities based on the conductivity of carbon include, having carbonized structures that serve as an anode and cathode. The material properties of the carbonized structures may be varied to provide different electrical or material properties. For example, one carbonized structure could incorporate oligodynamic particles on a surface to provide desired electrical effects, as well as oligodynamic inactivation of IAs. The structures can be connected to a power source like a battery which when switched causes current to flow. Filtration medium entrapping IAs may be disposed between the anode and cathode layers so that the current passing between helps inactivate the IAs. In another possibility embodiment, the anode and cathode layers are separated by a dielectric filter medium so that the layer can store an electrical charge. A discharge event will cause a capacitive, electrostatic effect across the dielectric layer intermediate the carbonized layers. If the dielectric layer is also the filter medium, the discharge will help disrupt entrapped IAs.

As another possibility, the energized structure could be conductive fabric that is attached to a power source, causing resistive heating in the layer. The layer could be different from or the same as the filtration medium. If different, it only needs to be sufficiently close for heat transfer.

A fifth functional feature is a hydrophobic outer surface that may actively repel mist, liquid droplets, and other moisture. In one of many possible examples, the outer layer is constructed with a manufactured fabric (e.g., polyester) that is impregnated with Durable Water Repellent (DWR) compounds. This DWR stops liquids at the outer surface of the fabric. Hydrophobic DWR, while somewhat durable, can wash out after many launderings but can be renewed with commonly available products (e.g., Nikwax). Another possibility is to make the outer surface from a textile made with hydrophobic yarns or fibers, e.g., expanded PTFE membrane. Such membrane could also be a backing to a more durable outer layer.

Another notable advantage of the inventive articles contemplated herein, unlike throw-away masks, the article may be constructed of durable materials that can be washed and reused. The fabrics and sewing materials used may be those that are common in many industries, including sports and casual clothing crafting.

FIGS. 1A-1B illustrates a two-layer filtration article wherein each layer may embody one or more of the functional features discussed herein. The article shown is just to illustrate a layering scheme, and the article may be embodied into any kind of PPE. FIGS. 2A-2B shows a filtration article in the form of mask or respirator. In this example, the filtration article includes an optional third layer sandwiched between the first and second layers. The inventive subject matter is not limited to an article of 1, 2 or 3 layers. It may have any greater number of layers that provide the desired functional features disclosed herein.

The layers contemplated herein may be discrete layers that are bonded, fused or otherwise joined together using known techniques. Or two or more layers may be unitary structures that are not formed of discrete structures. For example, knit and woven structures can be formed in multiple, seamlessly joined layers with the layers varying in terms of materials, yarn deniers, and/or crossover picks and/or loop densities. Similarly, non-wovens can have different layers monolithically formed by varying the size, denier, or material laid out or deposited in the formation process. Accordingly, a single physical layer may embody one or more functional features disclosed herein.

In the example embodiments of FIGS. 1A-2B, the two primary layers are an outer first layer and an inner second layer. Depending on use, the device may have one or more intermediate layers. FIGS. 2A-2B show an example intermediate layer (the third layer). For example, an intermediate layer may be a bonding layer, e.g., a thin polyester foam or glue, or a thermally fusible polymer layer. It may be a fabric-backing that adds strength and body. Some fusible polymer materials could serve as a bonding layer and a backing layer.

In the case of a mask or respirator like seen in FIGS. 2A-2B, the article forms a covering over at least the inhalation passages of the user's face, namely the nose and mouth. The covering may sit flush against the face, like a surgical mask. Or, as shown, it may have a three-dimensional shape that forms a void in front of the passages. Whatever form, the mask has perimeter that is intended to provide a seal against the skin so that environmental air, and whatever particles the air carries, must pass through the filtration layer(s) of the mask and any other desired functional layer. The covering may be a standalone article or it may part of a larger article. For example, it could be part of a full head covering, a jacket hood, and balaclava, a neck gaiter, etc. It could be a permanent part of any such article, or it could be removably, replaceably attached using any known system of joining, e.g., hook and loop fasteners, snaps, buttons, zippers and slide seals, glue, etc. When removably attached, the article to which it attaches can be configured with an opening design that fits against the mask so that there are no gaps around the mask, thereby providing a more effective environmental seal.

In the examples of FIGS. 2A-2B, the article is finished with a sewn binding material. The binding may contain an oligodynamic material so that there is better protection at all possible points of entry around the entire perimeter of the article.

Referring to FIGS. 2A-2B, the layers will be discussed in more detail. The discussion is intended to be non-limiting and only to illustrate one of many possible embodiments at a more detailed level
Layer 1:
This outside layer 14 in this example may have some or all the following components:
A) Textile base that may have carbon fiber, silver, copper or other oligodynamic material incorporated (e.g., fused, melted, admixed) into the fibers. The base or another component may also serve as a filtration medium to physically entrap particles. The fabric material and construction may be any one of those discussed elsewhere herein.
B) Durable water repellent finish on the exterior surface.
C) Surface treatment (e.g., spray, wet dip, etc.) on the exterior and/or interior surface or otherwise incorporating silver nanoparticles or similar oligodynamic material.
Layer 2:
This inside layer 12 in this example may have some or all the following components:
A) Textile base with carbon fiber.
B) Denier gradient moisture control textile. The fabric material and construction may be any one of those discussed elsewhere herein.
C) Textile or fibers thereof treated with or otherwise incorporating silver nanoparticles or other oligodynamic material, the same or different from the oligodynamic material on the first layer. The fabric material and construction may be any one of those discussed elsewhere herein.
Layer 3 (optional):
The middle or intermediate layer(s) 16 may contain some or all the following:
A) Polyester foam that may be treated with antimicrobial agents
B) Glue
C) Fusible material and/or backing material (e.g., a non-woven polymer material to bond adjacent layers or to give form to the mask)
D) Filtration medium to physically entrap particles.

Fabric materials to construct this invention can include but are not limited to: single product or blends of rayon, polyester, spandex, cotton, wool, elastane, polyamide, carbon fiber yarn, silk, cashmere, silver, copper, nylon, bamboo, and blends of any one or more of the foregoing materials. These fabrics may be used in any one or more layers of a filtration article, discussed in more detail below. Carbon fibers are often woven into athletic clothing for the wearer's comfort, and for this device, is appropriate for a face mask.

A carbon fiber is a long, thin strand of material. It may have a range of diameters. In some cases, expected to be suitable for use in the inventive subject, it has a diameter of about 0.005-0.010 millimeter. However, the inventive subject is not necessarily limited to that range and smaller or larger diameters may be useful, depending on the selected application. While not intending to be bound to any theories or principles, it is understood that the carbon atoms in carbon fiber are bonded together in microscopic crystals that are more or less aligned parallel to the long axis of the fiber. The crystal alignment makes the fiber incredibly strong for its size. Several thousand carbon fibers may be twisted together to form a yarn, which may be used by itself or woven into a fabric. The resulting fabric can be a drapable product suitable for garments and filtration articles. The carbon fibers may also be blended with other known yarn materials. As used herein, a carbon fiber yarn or textile or fabric refers to any such construct that has at least 2% carbon fiber or particles by volume. Carbon fiber yarns may be woven or knit into one of the fabric types discussed below.

Fabric types to construct this invention may include knit, woven, and/or non-woven textiles. The textiles may include but are not limited to: single knit, double knit, plaited, jersey, lame, mesh, tricot, fuse, ripstop, felting, laminating, bonding, canvas, pile, Jacquard, dobby, gauze, raschel tabby, twill, satin, buckram, cambric, casement, cheese cloth, chiffon, chintz, corduroy, crepe, denim, drill, flannel, gabardine, georgette, khadi, lawn, mulmul, muslin, poplin, sheeting, taffeta, tissue, velvet, mousseline, organdie/organza, leno, aertex, madras muslin, and aida.

The inventive subject matter may use a Durable Water Repellent (DWR) finish on outer layers to repel IAs contained in droplets expelled (e.g., by coughing or sneezing) into the environment surrounding a mask or other PPE article. Thus, the inventive subject matter provides a first layer of protection pre-filtration layer of protection that helps keep IAs from entering the mask. DWRs are non-polar or hydrophobic compositions. They are well-known and widely available in the general textile industry. They have been applied to a variety of textiles to inhibit water absorption. Because water is a polar molecule in the liquid phase it tends to clump into droplets on the hydrophobic DWR finish. These droplets are relatively easy to stop on a fabric face. In the vapor phase, water molecules are smaller and more energized therefore they can move easily through many textiles, both woven and non-woven, even those having a DWR finish. Thereby, articles according to the inventive subject may be finished with a DWR technology to repel droplets and other moisture while providing some venting (breathability) of moisture from the inward facing side of the article. An advantage of a DWR finish on an oligodynamic filtration article is it extends the articles usefulness and efficacy by eliminating moisture which can degrade the physical structure and features of the article.

Durable Water Repellent (DWR) finishes may include but are not limited to single products, polymers or blends using wax(s), oils, fluorocarbons, fluoropolymers, silicon, non-wax hydrocarbons, perfluorobutanesulfonic acid, perfluorooctanoic acid and related compounds. DWR application methods may include but are not limited to dipping, spraying, chemical vapor deposition and related techniques or combination of methods.

A variety of oligodynamic materials may be applied to or incorporated into textiles (or constituent fibers or yarns used to make the textiles) to kill, destroy, neutralize, or otherwise disrupt IAs like bacteria and viruses. These include, but are not limited to, silver, mercury, copper, iron, lead, zinc, bismuth, gold, aluminum, platinum, palladium, iridium, tin, and antimony. Oligodynamic metal salts can include, but are not limited to, silver acetate, silver carbonate, silver chloride, silver citrate, silver cyanide, silver hydroxide, silver nitrate, silver nitrite, silver oxide, silver phosphate, silver sulfate, mercury acetate, mercury carbonate, mercury chloride, mercury citrate, mercury cyanide, mercury hydroxide, mercury nitrate, mercury nitrite, mercury oxide, mercury phosphate, mercury sulfate, copper acetate, copper carbonate, copper chloride, copper citrate, copper cyanide, copper hydroxide, copper nitrate, copper nitrite, copper oxide, copper phosphate, copper sulfate, iron acetate, iron carbonate, iron chloride, iron citrate, iron cyanide, iron hydroxide, iron nitrate, iron nitrite, iron oxide, iron phosphate, iron sulfate, lead acetate, lead carbonate, lead chloride, lead citrate, lead cyanide, lead hydroxide, lead nitrate, lead nitrite, lead oxide, lead phosphate, lead sulfate, zinc acetate, zinc carbonate, zinc chloride, zinc citrate, zinc cyanide, zinc hydroxide, zinc nitrate, zinc nitrite, zinc oxide, zinc phosphate, zinc sulfate, bismuth acetate, bismuth carbonate, bismuth chloride, bismuth citrate, bismuth cyanide, bismuth hydroxide, bismuth nitrate, bismuth nitrite, bismuth oxide, bismuth phosphate, bismuth sulfate, gold acetate, gold carbonate, gold chloride, gold citrate, gold cyanide, gold hydroxide, gold nitrate, gold nitrite, gold oxide, gold phosphate, gold sulfate, aluminum acetate, aluminum carbonate, aluminum chloride, aluminum citrate, aluminum cyanide, aluminum hydroxide, aluminum nitrate, aluminum nitrite, aluminum oxide, aluminum phosphate, aluminum sulfate, platinum acetate, platinum carbonate, platinum chloride, platinum citrate, platinum cyanide, platinum hydroxide, platinum nitrate, platinum nitrite, platinum oxide, platinum phosphate, platinum sulfate, palladium acetate, palladium carbonate, palladium chloride, palladium citrate, palladium cyanide, palladium hydroxide, palladium nitrate, palladium nitrite, palladium oxide, palladium phosphate, palladium sulfate, iridium acetate, iridium carbonate, iridium chloride, iridium citrate, iridium cyanide, iridium hydroxide, iridium nitrate, iridium nitrite, iridium oxide, iridium phosphate, iridium sulfate, tin acetate, tin carbonate, tin chloride, tin citrate, tin cyanide, tin hydroxide, tin nitrate, tin nitrite, tin oxide, tin phosphate, tin sulfate, antimony acetate, antimony carbonate, antimony chloride, antimony citrate, antimony cyanide, antimony hydroxide, antimony nitrate, antimony nitrite, antimony oxide, antimony phosphate, antimony sulfate, or combinations thereof. Suitable oligodynamic metals or oligodynamic metal salts could be readily obtained or prepared by persons of skill in the art and incorporated into a filtration article as described herein.

Contrary to conventional thinking, in some applications, depending on the oligodynamic material, some moisture in a layer of a mask or other article with oligodynamic material may be desirable. For example, the moisture may help activate an oligodynamic metal into a more active form or help disperse it through a layer or layers of the article for wider distribution and more effective action. To provide both user comfort and such moisture activation, the internal portion of the mask may use a moisture management structure like a denier differential or carbonized fabric to move moisture away from the face or other body part to a more outward portion where oligodynamic material is to be moisture activated. For example, the moisture management feature may be present at an internal surface and the oligodynamic feature may be at an intermediate layer or portion.

U.S. Pat. No.: 8,183,167 to Delattre et al is incorporated here by reference. The Abstract reads: “Substrates that exhibit antimicrobial and/or antifungal characteristics that persist through the useful life of the substrate, and more particularly textile substrates infused with or covalently bound to well-dispersed antimicrobial nanoparticles, such as silver and/or copper nanoparticles, which exhibit persistent and demonstrable bactericidal, bacteriostatic, fungicidal, fungistatic behavior through numerous wash cycles. Methods of manufacturing such substrates are also provided.”

The oligodynamic material may be in the nature of nanoparticles. A nanoparticle is usually defined as a particle whose diameter is between 1 and 100 nanometers. Nanoparticles are usually distinguished from “fine particles”, sized between 100 and 2500 nanometers, and “coarse particles”, ranging from 2500 to 10,000 nanometers. They are a subclass of the colloidal particles, which are usually understood to range from 1 to 1000 nanometers. The properties of nanoparticles often differ markedly from those of larger particles of the same substance. Since the typical diameter of an atom is between 0.15 and 0.6 nm, a large fraction of the nanoparticle's material lies within a few atomic diameters from its surface. Therefore, the properties of that surface layer may dominate over those of the bulk material. This effect is particularly strong for nanoparticles dispersed in a medium of different composition, since the interactions between the two materials at their interface also becomes significant. Ref: Batista, Carlos A. Silvera; Larson, Ronald G.; Kotov, Nicholas A. (9 Oct. 2015). “Nonadditivity of nanoparticle interactions”. Science. 350 (6257): 1242477. doi:10.1126/science.1242477. ISSN 0036-8075. PMID 26450215.

A benefit of nanoparticles is that millions or more of them can be applied to and impregnated into a square meter of fabric. For example, silver nanoparticles have a very high surface area, thus the chance of contact with an IA is very high. The !As may contact fixed silver-based oligodynamic materials fixed to the textile substrate or the IA may encounter metallic ions that are generated when the metal is exposed to moisture. In a mask, this moisture would come from the person exhaling through it.

As discussed above, a filtration article according to the inventive subject matter may be composed of one or more layers of specialized fabrics that provide multiple functions alone or collectively.

When worn on the face, the user breathes in and out through the fabric layer or layers including oligodynamic material, IA in the inhaled air will contact the oligodynamic materials that are on or in the fabric. Laboratory tests have shown that over 600 species of bacteria and viruses can be neutralized or destroyed by encountering metallic elements (e.g., copper, silver).

Persons skilled in the art will recognize that many modifications and variations are possible in the details, materials, and arrangements of the parts and actions which have been described and illustrated in order to explain the nature of the inventive subject matter, and that such modifications and variations do not depart from the spirit and scope of the teachings and claims contained therein.

All patent and non-patent literature cited herein is hereby incorporated by references in its entirety for all purposes.

As used herein, “and/or” means “and” or “or”, as well as “and” and “or.” Moreover, any and all patent and non-patent literature cited herein is hereby incorporated by references in its entirety for all purposes.

The principles described above in connection with any particular example can be combined with the principles described in connection with any one or more of the other examples. Accordingly, this detailed description shall not be construed in a limiting sense, and following a review of this disclosure, those of ordinary skill in the art will appreciate the wide variety of systems that can be devised using the various concepts described herein. Moreover, those of ordinary skill in the art will appreciate that the exemplary embodiments disclosed herein can be adapted to various configurations without departing from the disclosed principles.

The previous description of the disclosed embodiments is provided to enable any person skilled in the art to make or use the disclosed innovations. Various modifications to those embodiments will be readily apparent to those skilled in the art, and the generic principles defined herein may be applied to other embodiments without departing from the spirit or scope of this disclosure. Thus, the claimed inventions are not intended to be limited to the embodiments shown herein, but are to be accorded the full scope consistent with the language of the claims, wherein reference to an element in the singular, such as by use of the article “a” or “an” is not intended to mean “one and only one” unless specifically so stated, but rather “one or more”.

All structural and functional equivalents to the elements of the various embodiments described throughout the disclosure that are known or later come to be known to those of ordinary skill in the art are intended to be encompassed by the features described and claimed herein.

Moreover, nothing disclosed herein is intended to be dedicated to the public regardless of whether such disclosure is explicitly recited in the claims. No claim element is to be construed as “a means plus function” claim under US patent law unless the element is expressly recited using the phrase “means for” or “step for”.

The inventors reserve the right to claim, without limitation, at least the following subject matter.

1. A filtration article for using in personal protective equipment (“PPE”), comprising:
one or more layers, the article having an external side for facing side the environment, and an internal side for facing the body of a user, and wherein the article is air permeable but has a porosity sufficient to entrap and thereby filter selected infectious agents (IAs), and wherein the one or more layers comprise structures that provide two or more of the following functional features selected from the group of:
(1) a structure with oligodynamic materials for inactivating the selected IAs;
(2) a structure of carbon fibers or particles for moisture management and/or conductive cooling;
(3) a knit or woven structure having yarns in a denier gradient that causes wicking of water from the internal side of the mask toward the external side;
(4) an energized or energizable structure that subjects entrapped IAs to current, charged particles, electrostatic discharge, resistive heating, IR heating and/or disruptive electromagnetic energy; and
(5) a hydrophobic structure forming an external surface of the external side for repelling droplets and other moisture from absorbing into the surface.

2. The article of claim 1 wherein the selected functional features include an oligodynamic structure.

3. The article of claim 2 wherein the oligodynamic structure comprises a textile structure.

4. The article of claim 3 wherein the structure comprises a knit, woven or non-woven structure comprising yarns or fibers.

5. The article of claim 4 wherein the yarns or fibers have coated or embedded oligodynamic particles sufficiently exposed so that they can inactivate the selective IAs.

6. The article of claim 5 wherein the particles comprise silver or copper particles or salts or other compositions thereof.

7. The article of claim 5 wherein the particles comprise nanoscale particles.

8. The article of claim 2 further including the denier gradient structure.

9. The article of claim 2 further including the hydrophobic structure.

10. The article of claim 9 wherein the hydrophobic structure comprises a knit, woven or non-woven textile that has a DWR finish.

11. The article of claim 9 wherein the hydrophobic structure comprises a knit, woven or non-woven textile comprising yarns or fibers that are inherently hydrophobic.

12. The article of claim 2 further including a first carbon fiber or carbon particle structure (a “carbonized structure”).

13. The article of claim 12 wherein the carbonized structure comprises the moisture management and/or conductive cooling structure.

14. The article of claim 12 wherein the carbonized structure comprises the energized or energizable structure.

15. The article of claim 12 further comprising a second carbonized structure, the first and second carbonized structures being on different layers.

16. The article of claim 12 wherein the carbonized structure comprises both the (1) moisture management and/or conductive cooling structure and (2) the energized or energizable structure.

17. The article of claim 12 wherein the carbonized structure comprises a knit or woven structure comprising carbon fibers or particles.

18. The article of claim 1 wherein the functional features are each provided in the same or a different layer.

19. The article of claim 1 wherein there are a plurality of oligodynamic structures, each on different layers.

20. The article of claim 13 wherein the oligodynamic structure and the structure for moisture management and/or conductive cooling are structures each forming a different layer.

21. The article of claim 12 wherein the oligodynamic structure and the energized or energizable structure are structures each forming a different layer.

22. The article of claim 1 wherein the article comprises at least three of the functional features.

23. The article of claim 1 wherein the article comprises at least four of the functional features.

24. The article of claim 1 wherein the article comprises all five of the functional features.

25. The article of claim 5 wherein the article comprises a mask or respirator sized and shaped to surround and cover at least the inhalation passages of an intended user.

26. The article of claim 25 wherein the article is made of materials and has a construction sufficiently durable to withstand at least 25 cycles of washing or laundering under conditions typical of home laundering.

27. The article of claim 5 wherein a moisture management structure is an inward layer and oligodynamic structure is a more outward layer, the moisture management layer serving to move moisture towards the oligodynamic layer, the oligodynamic layer being more active when receiving the moisture.

28. The article of claim 5 wherein the article has a porosity sufficient to entrap viruses, including corona viruses.
